(19) United States
(12) Patent Application Publication
(10) Pub. No.: US 2017/0165380 A1
(43) Pub. Date: Jun. 15, 2017

(54) Nanosensor Compositions and Methods of use Thereof

(71) Applicant: Northeastern University, Boston, MA (US)

(72) Inventors: Heather A. Clark, Lexington, MA (US); Yi Luo, Malden, MA (US)

(21) Appl. No.: 15/373,096

(22) Filed: Dec. 8, 2016

Related U.S. Application Data
(60) Provisional application No. 62/266,559, filed on Dec. 11, 2015.

(51) Int. Cl. A61K 49/18; A61K 49/08

(52) U.S. Cl. CPC A61K 49/1839; A61K 47/48246; A61K 47/489; A61K 49/085

(57) ABSTRACT

Disclosed are compositions comprising a plastic polymer core, a shell surrounding the plastic polymer core, a ligand comprising a binding moiety, an enzyme capable of catalyzing the hydrolysis of a target compound, and a contrast agent, as well as methods of use of such compositions.

### RELATED APPLICATION

This application claims the benefit of priority to U.S. Provisional Patent Application Ser. No. 62/266,559, filed Dec. 11, 2015.

### BACKGROUND

The development of novel imaging tools for real-time detection of molecular neural events, namely neurotransmitter release, is fundamental for studying the basis of brain function and disease. Neurotransmitters, such as acetylcholine, are an important class of messenger molecules that regulate chemical communication between cells in the brain. Signaling can be localized to a pair of cells, as occurs across a synaptic cleft via synaptic transmission. Signaling can also occur by extrasynaptic or volume transmission where neurotransmitters spill over and diffuse away from the site of release. In particular, the cholinergic system, which uses acetylcholine almost exclusivity to send messages between neurons, is one of the most important modulatory neurotransmitter systems in the brain, in which both synaptic and volume transmission govern activities that depend on selective attention, formation of working memories and cognitive behavior. Additionally, perturbations of the cholinergic system are implicated in schizophrenia, depression and Alzheimer's disease. As such, the ability to monitor spatiotemporal dynamics of neurotransmitter release is crucial for understanding the basis of brain function.

Imaging tools for detection of neurotransmitter release in the central nervous system can facilitate the understanding of neural signaling events critical to brain function and disease.

### SUMMARY

In some aspects provide herein are methods and compositions for sensing analytes. In some embodiments, the compositions comprises a plastic polymer core, a shell surrounding the plastic polymer core, a ligand comprising a binding moiety, an enzyme capable of catalyzing the hydrolysis of a target compound, and a contrast agent. In some embodiments, the shell comprises a plurality of amphiphilic lipids, each of which comprises a hydrophobic end and a hydrophilic end; and the hydrophobic ends of the amphiphilic lipids are linked to the plastic polymer core. In some embodiments, the binding moiety is capable of binding to a binding site present on a transmembrane receptor (e.g., an acetylcholine receptor). In some embodiments, the ligand (e.g., a protein, such as bungarotoxin), the enzyme, and the contrast agent are conjugated to the hydrophilic ends of the amphiphilic lipids. In some embodiments, the compositions are used in fMRI.

In some aspects, provided herein are methods related to imaging a target compound in a subject by administering to the subject an effective amount of a composition (e.g., a composition disclosed herein), and obtaining a functional magnetic resonance image (fMRI) of the subject, thereby imaging the target compound. In some embodiments, the target compound is a neurotransmitter (e.g., acetylcholine). In some embodiments, the fMRI image is of the subject's brain.

### BRIEF DESCRIPTION OF THE DRAWINGS

The application file contains at least one drawing executed in color. Copies of this application publication with color drawing(s) will be provided by the U.S. Patent and Trademark Office upon request and payment of the necessary fee.

FIG. 1 shows a schematic of a nanosensor structure and its mechanism. Part A illustrates the nanosensor has a hydrophobic PVC core coated by an amphiphilic DSPE-PEG-lipid shell. Both pH-sensitive contrast agent and butyrylcholinesterase (BuChE) are conjugated to hydrophilic ends of DSPE-PEG-lipids. Part B shows the BuChE catalyzes the hydrolysis of acetylcholine to choline and acetic acid, and the resulting drop in local pH triggers a conformational switch of the contrast agent: There is one more water molecule coordinated to one Gd(III) chelate in acidic conditions compared to its structure in basic conditions, which leads to increased T₁ relaxation rate (1/T₁) of the contrast agent. Compounds 1: pH-sensitive Gd-PEG, 2: BuChE conjugated-PEG, 3: acetylcholine, 4: choline, 5: acetic acid.

FIG. 2 shows the characterization, in vitro calibration and selectivity of nanosensors. Dynamic light scattering (DLS) analysis showing size distribution of nanosensors. Part A shows results without conjugation to BuChE, and Part B shows results with conjugated to BuChE. Part C shows TEM image of nanosensors stained with Nanovan. Part D and E shows in vitro calibration of nanosensor. Nanosensors were exposed to solutions of 0, 10, 50, 100, 500 and 1000 μM of acetylcholine. Part D shows higher concentrations of acetylcholine led to brighter MR images. The blue square indicates the ROI. Part E shows 1/T₁ of nanosensors was enhanced when higher concentrations of acetylcholine were present. Part F shows the selectivity towards acetylcholine of nanosensors. Nanosensors were exposed to PBS only or to solutions of acetylcholine (0.5 mM), glutamate (5 mM), dopamine (5 mM), GABA (5 mM) and glycine (5 mM). 1/T₁ in solutions of acetylcholine was significantly higher than other groups (one-way ANOVA, *P<0.005, for n=3). The 1/T₁ in Part E and F were calculated from an average T₁ of three independent measurements. Error bars in Part E and F were calculated from standard deviation (S.D.) of T₁ using error propagation.

FIG. 3 shows in vivo sensor contrast. Part A shows a schematic diagram of placement of cannula and ACh nanosensor infusion (blue) to the rat mPFC, and MM data acquired from sensor encompassing slices. Part B shows coronal brain slice (bregma: +2.8 mm) of pre-(top panel) and post-nanosensor injection (bottom). Arrowhead (red) indicates the position of cannula, and the circle (blue) defines the ROI for analysis. Part C shows ROI-averaged MR signal intensity showing increase in 1/T₁ for >60% in the post-nanosensor injection in comparison to pre-injection slice. Error bars were calculated from S.D. of T₁ using error propagation. Diagram adapted from Paxinos and Watson.

FIG. 4 shows Acetylcholine detection in vivo. Part A shows an experimental procedure. Concomitant nanosensor and drug (clozapine) were delivered respectively through cannula and subcutaneous injections of clozapine to the back of rats, followed by three consecutive MR scans 30 min apart denoted, t=0, 30, 60 min. Part B shows coronal brain slices showing time-courses of acetylcholine detection from pre-delivery to 60 min. In the experimental group, nanosensors were injected through the cannula, and clozapine was administered subcutaneously (top panels, n=6). In the control group, nanosensors were injected without clozapine administration (bottom panels, n=6). Part C shows distinct acetylcholine signal changes accompanied by a significant increase in 1/T₁ (P=0.018; Student's t-test) observed in the experimental group (blue, n=6) at 60 min compared to the control group (grey, n=6). Error bars were calculated from S.D. of normalized T₁ using error propagation.

FIG. 5 shows structure and ¹H NMR spectrum of pH-sensitive chelator. Structure (inset) and the spectrum was obtained from a Varian Inova 500 MHz NMR spectrometer. ¹H NMR (500 MHz, D₂O): δ=8.35 ppm (s, 1H), 8.22 ppm (m, 1H), 7.01 ppm (m, 1H), 4.97 ppm (m, 1H), 3.66 ppm (s, 2H), 3.36 ppm (m, 16H), 3.05 ppm (m, 6H).

FIG. 6 shows in vitro calibration of nanosensor to pH. Response of nanosensors to varying pH was examined in PBS at pH 6, 6.5, 7, 7.5 and 8. The 1/T₁ was calculated from an average T₁ of three independent measurements. Error bars were calculated from S. D. of T₁ using error propagation.

FIG. 7 shows histology representative photomicrographs showing placement of cannula in the rat cortex. Coronal rat brain sections in the bottom left panel shows choline acetyltransferase (ChAT) immunoreactivity and cresyl violet (Nissl) histological staining (bottom right panel) at the level of mPFC. Diagram adapted from Paxinos and Watson.

FIG. 8 shows pH-NP control in vivo. Identical MR scanning procedures were conducted in a new cohort (pH-NP group, red line, n=3) to study the localized effects of enzymatic hydrolysis of acetylcholine to trigger changes in 1/T₁. In this group, nanosensors without conjugated enzymes, i.e., pH-sensitive nanoparticles (pH-NP) were delivered along with clozapine administration identical to the experimental group (blue line). After 1 h, 1/T₁ decreased by >15% in comparison to the experimental group, indicating discrete detection of acetylcholine driven by local enzymatic hydrolysis, and not due to the effects of clozapine. Error bars were calculated from S.D. of normalized T₁ using error propagation.

FIG. 9 shows a conceptualization of using real-time nanosensors to measure acetylcholine release around the synaptic cleft (red sphere).

FIG. 10 shows one water molecule coordinates on Gd under basic conditions, while two water molecules coordinate on Gd under acidic conditions. The change in water coordination results in a change in T1 signal.

FIG. 11 shows calibration of MR nanoparticles using absorbance (black) and MR (red) methods.

FIG. 12 illustrates an exemplary embodiment of the invention and the structure of enzyme tagged nanoparticle.

FIG. 13 shows an image of acetylcholine-selective nanosensors in acetylcholine solution in a 96-well plate.

FIG. 14 shows a T1 calibration against acetylcholine from the data in FIG. 13.

FIG. 15 shows co-injection of nanosensors and acetylcholine (left circle) gives a brighter image than control (right circle).

FIG. 16 shows the position and orientation of cannula in the rat medial prefrontal cortex (green box).

FIG. 17 shows pre-scan without nanosensor (1); right after injection of nanosensor (2); 20 min after injection (3); 40 min after injection (4).

FIG. 18 shows region of interest (ROI) at injection site (Green) and control ROI (yellow)(A). Normalized T1 difference between injection site and control site in experiment group and control group (B).

FIG. 19 illustrates an exemplary embodiment of the invention.

### DETAILED DESCRIPTION

In some aspects, provided herein compositions comprising a plastic polymer core, a shell surrounding the plastic polymer core, a ligand comprising a binding moiety, an enzyme capable of catalyzing the hydrolysis of a target compound, and a contrast agent. In some embodiments, the shell comprises a plurality of amphiphilic lipids, each of which comprises a hydrophobic end and a hydrophilic end; and the hydrophobic ends of the amphiphilic lipids are linked to the plastic polymer core. In some embodiments, the binding moiety is capable of binding to a binding site present on a transmembrane receptor. The ligand, the enzyme, and the contrast agent may be conjugated to the hydrophilic ends of the amphiphilic lipids. Also provided herein are methods of using the compositions disclosed herein. In some embodiments, the methods provided herein relate to imaging a target compound in a subject by administering to the subject an effective amount of a composition (e.g., a composition disclosed herein), and obtaining a functional magnetic resonance image (fMRI) of the subject.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

A “fluorophore” is a molecule that absorbs light at a characteristic wavelength and then re-emits the light most typically at a characteristic different wavelength. Fluorophores are well known to those of skill in the art and include, but are not limited to rhodamine and rhodamine derivatives, fluorescein and fluorescein derivatives, coumarins and chelators with the lanthanide ion series. A fluorophore is distinguished from a chromophore which absorbs, but does not characteristically re-emit light.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

### OVERVIEW

The invention disclosed herein relates, in part, to the development of a nanosensor for detection of compounds (e.g., such as neurotransmitters, such as acetylcholine) in a subject using magnetic resonance imaging (MRI). In some embodiments, the compounds (e.g., target compounds) are present in the brain of the subject. MRI is an attractive tool for in vivo sensing as it can be applied noninvasively, in living organisms at any depth, with relatively high spatial and temporal resolution. MRI creates an image by applying a radio-frequency pulse to a subject to disturb steady-state proton magnetization. The perturbation of protons produces an image based on signal contrast. The signal contrast results from interactions between applied radio-frequency and magnetic field gradient pulses, and the distribution-relaxation kinetics of nuclear spins in a specimen, most commonly water protons. Signal can be further manipulated using contrast agents, by shortening longitudinal (T₁) or transverse (T₂) relaxation times to produce an increase or decrease in signal intensity, respectively. The efficiency of contrast agents to decrease T₁ is referred to relaxivity (r₁). Contrast agents with higher r₁ lead to lower T₁ of chelated water protons. The early and widely implemented MRI contrast agents are small-molecule chelates that incorporate paramagnetic ions that alter T₁, such as gadolinium (Gd³⁺) or manganese (Mn²⁺ or Mn³⁺).

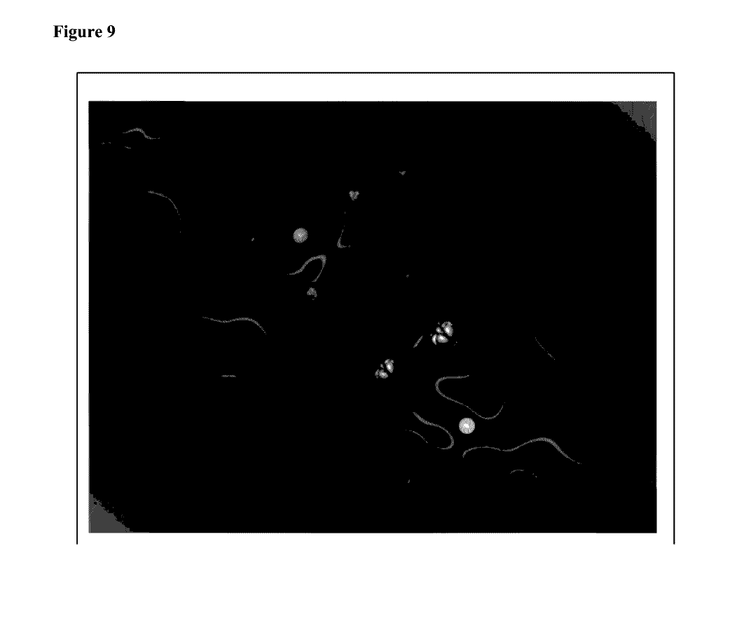

Current techniques for imaging neurotransmitters suffer from low spatial resolution and difficulties in accessing central synapses (FIG. 9). For this reason, a variety of techniques that have been developed for imaging and tracking neurotransmitters demonstrate limited in vivo applications. For example, fluorescence imaging in vivo suffers from inadequate optical access, and optical aberration. Also, most fluorescent probes are not specifically designed for tagging neurotransmitters and are not responsive to concentration changes in the physiological range.

Imaging techniques, such as functional MRI (fMRI), connect the structural and functional information for neural imaging in living organisms, but the accessible chemical information is very limited. A variety of fMRI methods have been established, including hemodynamic fMRI, metal ion fMRI, and pH fMRI. fMRI uses radio waves to scan tissue, and offers better tissue depth penetration relative to traditional optic imaging. The use of contrast agents such as chelated gadolinium ions [Gd (III)] can reduce the longitudinal relaxation time (T1), and provide a high contrast image. The ability of contrast agents to differentiate T1 of samples from background water is referred to as relaxivity 1 (r1), and a larger r1 yields better contrast. The r1 of Gd (III)-based contrast agents can be determined by multiple factors, including the number of water molecules that are coordinated on Gd. Previous attempts to modify contrast agents have covalently conjugated them to nanomaterials such as nanodiamonds or nanotubes. By decreasing the tumbling rate, these designs help to increase the r1 of contrast agents.

### Nanosensor Composition

In some aspects, provided herein are compositions comprising a plastic polymer core, a shell surrounding the plastic polymer core, a ligand comprising a binding moiety, an enzyme capable of catalyzing the hydrolysis of a target compound, and a contrast agent. In other aspects, provided herein are methods of obtaining as image of a target compound by administering the composition to the subject and obtaining an fMRI image of the subject.

In some embodiments, the shell comprises a plurality of amphiphilic lipids, each of which comprises a hydrophobic end and a hydrophilic end; and the hydrophobic ends of the amphiphilic lipids are linked to the plastic polymer core. In some embodiments, the binding moiety is capable of binding to a binding site present on a transmembrane receptor. The ligand, the enzyme, and the contrast agent may be conjugated to the hydrophilic ends of the amphiphilic lipids. Conjugating the ligand, enzyme or the ligand to the polymer plastic core can be done by any technique known in the art. In some embodiments, copper-free click chemistry may be used. Click chemistry involves the rapid generation of compounds by joining small units together via heteroatom links (C—X—C). For example, an azide-modified enzyme may be conjugated to the DSPE-PEG-dibenzocyclooctyne (DSPE-PEG-DBCO) lipid by incubation in low temperatures. In some embodiments, the contrast agent was conjugated to shell via an amide bond.

In some embodiments, the plastic polymer core is consists of thermostable plastic (e.g., polyvinyl chloride (PVC)). In some embodiments, the plurality of amphiphilic lipids comprises 1,2-distearoyl-sn-glycero-3-phosphoethanolamine-N-(polyethyleneglycol) (DSPE-PEG). In some embodiments, the pH sensitive contrast agents are covalently linked to the amphiphilic lipids.

In some embodiments, the transmembrane receptor is located on a synaptic membrane (e.g., an acetylcholine receptor). In some embodiments, the ligand is a ligand for a transmembrane receptor (e.g., an acetylcholine receptor). The ligand may be a protein, such as bungarotoxin. Bungarotoxin is a protein that can specifically bind to nicotinic receptors in the synaptic cleft. In some embodiments, the bungartoxin may target and bind the neuronal synapse (i.e., a transmembrane receptor on the synapse) of the subject, preventing dispersion of the compositions. In some embodiments, a segment of bungarotoxin can be attached to the plastic polymer core. In some embodiments, the bungarotoxin is attached to the plastic polymer core via conjugation to the lipid shell surrounding the core. Ligands may be conjugated to the lipids using any technique known in the art. For example, bungarotoxin can be tagged to DSPE-PEG carboxylate via EDC/NHS chemistry. Modified DSPE-PEG can be used to fabricate nanosensors as stated in example section.

The ligand may be acetylcholine. Acetylcholine is a common signaling molecule at the neuromuscular junction, in the autonomic nerve system, and in the brain. For example, cells in the basal forebrain and pontomesencephalic cholinergic complexes (i.e., complexes that use acetylcholine as a neurotransmitter) in the brain are associated with sleep-wake regulation, learning, and memory. Impairment of cholinergic function is believed to be involved in Alzheimer's disease and Lewy body disease. Acetylcholine is released from vesicles at the pre-synaptic membrane and binds to nicotinic or muscarinic receptors on the post-synapse membrane, where it is rapidly hydrolyzed by cholinesterase into acetic acid and choline.

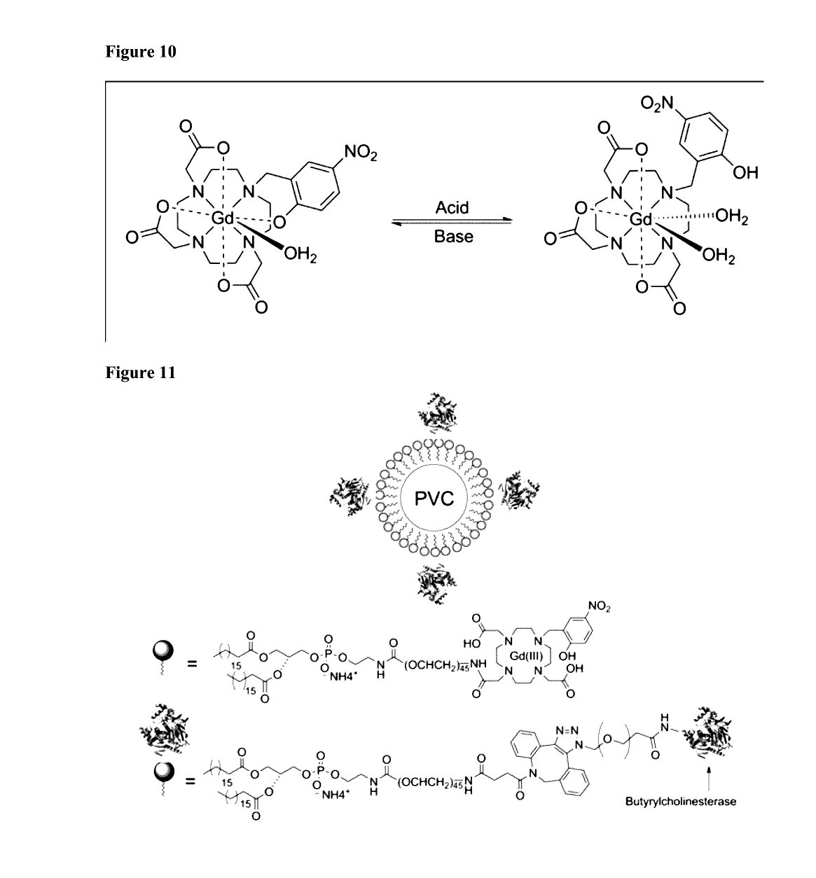

The compositions disclosed herein may comprise an enzyme. In some embodiments, the enzyme is a cholinesterase (e.g. a butyrylcholinesterase or an acetylcholinesterase). The compositions disclosed herein may comprise contrast agents to enhance contrast in fMRI, as well as may be used for analyte detection. The contrast agent may comprise gadolinium (Gd). Non-limiting examples of Gd-comprising contrast agents are gadoterate, adodiamide, gadobenate, gadopentetate, gadoteridol, gadoversetamide, gadoxetate, gadobutrol, gadoterate, gadodiamide, gadobenate, gadopentetate, gadoteridol, gadofosveset, gadoversetamide, gadoxetate, and gadobutrol. In some embodiments, the contrast agent comprises 1,4,7,10-tetraazacyclododecane-1,4,7,10-tetraacetic acid (DOTA). In other embodiments, the contrast agent is DOTA-Gd. The contrast agent may be GdNP-DO3A (gadolinium 1-methlyene-(p-NitroPhenol)-1,4,7,10-tetraazacycloDOdecane-4,7,10-triAcetate). In some embodiments, the contrast agent is pH sensitive. For example, 1,4,7,10 tetraazacyclododecane-1,4,7,10-tetraacetic acid (DOTA) may be used for pH sensing. This molecule contains a p-nitrophenol on a twelve-member ring. Under basic conditions, only one water molecule is involved in the coordination, while under acidic conditions, two water molecules will coordinate to Gd, leading to changes in r₁ and T₁ (FIG. 10). The contrast agent may be an iron oxide, iron platinum, or manganese contrast agent. The contrast agent may be protein contrast agent. The contrast agent should be capable of providing appropriate response to whatever MRI resolution is desired and whatever MRI intensity is used. Additional contrast agents may be found in U.S. Pat. No. 6,321,105, and U.S. Patent Publication US 2015/0202330, each of which is incorporated in their entirety.

In some embodiments, the composition is substantially spherical. The composition may have a diameter of about 10 nm or less, about 20 nm or less, about 30 nm or less, about 40 nm or less, about 50 nm or less, about 60 nm or less, about 70 nm or less, about 80 nm or less, about 90 nm or less, about 100 nm or less, about 110 nm or less, about 120 nm or less, about 130 nm or less, about 140 nm or less, about 150 nm or less, or about 160 nm or less.

In some embodiments, the core is seeded with silica. In some embodiments, the composition has a soft core structure that is rigidized with a silane reagent, while poly(ethylene glycol) chains form an outer shell. Examples of fabrication of soft core structures can be found in Wang et al. J Am Chem Soc. (2011) 134:41.

In some embodiments, the composition further comprises a fluorescent dye. The fluorescent dye may be a derivative of rhodamine, erythrosine, or fluorescein.

The fluorescent dye may be a xanthene derivative dye, an azo dye, a biological stain, or a carotenoid. The xanthene derivative dye may be a fluorene dye, a fluorone dye, or a rhodole dye. The fluorene dye may be a pyronine dye or a rhodamine dye. The pyronine dye may be chosen from pyronine Y and pyronine B. The rhodamine dye may be rhodamine B, rhodamine G and rhodamine WT. The fluorone dye may be fluorescein or fluorescein derivatives. The fluorescein derivative may be phloxine B, rose bengal, or merbromine. The fluorescein derivative may be eosin Y, eosin B, or erythrosine B. The azo dye may be methyl violet, neutral red, para red, amaranth, carmoisine, allura red AC, tartrazine, orange G, ponceau 4R, methyl red, or murexide-ammonium purpurate.

Exemplary fluorescent dyes include, but not limited to Methylene Blue, rhodamine B, Rose Bengal, 3-hydroxy-2, 4,5, 7-tetraiodo-6-fluorone, 5, 7-diiodo-3-butoxy-6-fluorone, erythrosin B, Eosin B, ethyl erythrosin, Acridine Orange, 6′-acetyl-4, 5, 6, 7-tetrachloro-2′,4′, 5′, 6′, 7′-tetraiodofluorescein (RBAX), fluorone, calcein, carboxyfluorescein, eosin, erythrosine, fluorescein, fluorescein amidite, fluorescein isothiocyanate, indian yellow, merbromin, basic red 1, basic red 8, solvent red 45, rhodamine 6G, rhodamine B, rhodamine 123, sulforhodamine 101, sulforhodamine B, and Texas Red (sulforhodamine 101 acid chloride).

### Methods

In some aspects, provided herein are methods of imaging a target compound in a subject, comprising administering an effective amount of to the subject a composition disclosed herein, and obtaining a functional magnetic resonance image (fMRI) of the subject, thereby imaging the target compound. In some embodiments, the target compound is a neurotransmitter, such as acetylcholine. In some embodiments, the fMRI image is of the subject's brain.

The compositions disclosed herein may be administered through any mode of administration. In some aspects, the compositions may be administered intracranially. In some aspects, the compositions are suitable for parenteral administration. These compositions may be administered, for example, intraperitoneally, intravenously, or intrathecally. In some aspects, the compositions are injected intravenously. In some embodiments, the compositions may be administered enterally or parenterally. Compositions may be administered subcutaneously, intravenously, intramuscularly, intranasally, by inhalation, orally, sublingually, by buccal administration, topically, transdermally, or transmucosally. Compositions may be administered by injection. In some embodiments, compositions are administered by subcutaneous injection, orally, intranasally, by inhalation, or intravenously. In certain embodiments, the compositions disclosed herein are administered by subcutaneous injection.

In some embodiments, the subject may be a mammal. In some embodiments, the subject may be a rodent, lagomorph, feline, canine, porcine, ovine, bovine, equine, or primate. In certain embodiments, the subject is a human. In some embodiments, the subject may be a female or male. In some embodiments, the subject may be an infant, child, or adult.

### Composition Delivery

In some aspects disclosed herein are compositions for sensing analytes, such as neurotransmitters. In some embodiments, the compositions disclosed herein comprise modifications to facilitate the composition across the blood brain barrier (BBB). In some embodiments, the compositions disclosed herein are administered to the subject under conditions that allow passage of the compositions across the BBB.

The BBB is formed by special tight junctions between the epithelial cells that surround the brain tissue. All tissue is separated by this layer of epithelial cells, however only the brain epithelial cells have these tight junctions that do not allow larger molecules to pass between them. One solution is to physically or chemically modify the composition to pass from the blood stream into the cranium. Another solution is to increase permeability of the BBB. By decreasing the restrictiveness of the barrier, it is much easier to get a molecule to pass through it. Compounds that increase the permeability of the BBB are known as receptor-mediated permabilitizers. These drugs increase the permeability of the blood-brain barrier temporarily by increasing the osmotic pressure in the blood which loosens the tight junctions between the endothelial cells. By loosening the tight junctions, injection of compositions through an IV can take place and be effective to enter the brain. In some embodiments, the compositions disclosed herein are administered concurrently with receptor-mediated permabilitizers. In other embodiments, the subject may be administered a receptor-mediated permabilitizer and compositions disclosed herein at different time points. For example, the subject may be administered a receptor-mediated permabilitizer a certain period of time prior to administration of the composition in order to allow the receptor-mediated permabilitizer to act on the BBB membrane.

The compositions disclosed herein may further comprise a nanoparticle delivery system to cross the BBB. These are systems where the (i.e., a nanoparticle comprising a composition disclosed herein) is bound to a nanoparticle capable of traversing the blood-brain barrier, such as Human Serum Albumin (HSA). The main benefit of this is that particles made of HSA are well tolerated without serious side effects. In addition, albumin functional groups can be utilized for surface modification that allows for specific cell uptake.

In some embodiments, the compositions disclosed herein are administered with ultrasound, such as focused ultrasound. In some embodiments, the compositions described herein may be administered to the subject using ultrasound (e.g., focused ultrasound). In some embodiments, the compositions are enclosed in microbubbles. Microbubbles are small “bubbles” of mono-lipids that are able to pass through the blood-brain barrier. They form a lipophilic bubble that can easily move through the barrier. Focused ultrasound increases the permeability of the blood-brain barrier by causing interference in the tight junctions in localized areas. This combined with the microbubbles allows for a very specific area of diffusion for the microbubbles, because they can only diffuse where the ultrasound is disrupting the barrier. The hypothesis and usefulness of these is the possibility of loading a microbubble with a composition described herein to diffuse through the barrier and target a specific area. There are several important factors in making this a viable solution for drug delivery. The first is that the loaded microbubble must not be substantially greater than the unloaded bubble. This ensures that the diffusion will be similar and the ultrasound disruption will be enough to induce diffusion.

Methods of using ultrasound to permeate the BBB maybe found in U.S. Patent Application US 2009/0005711, U.S. Pat. No. 6,514,221, and U.S. Pat. No. 7,344,509, each of which is hereby incorporated in its entirety.

### EXAMPLES

The present examples are non-limiting implementations of the use of the present technology.

### Example 1: Nanosensor Development

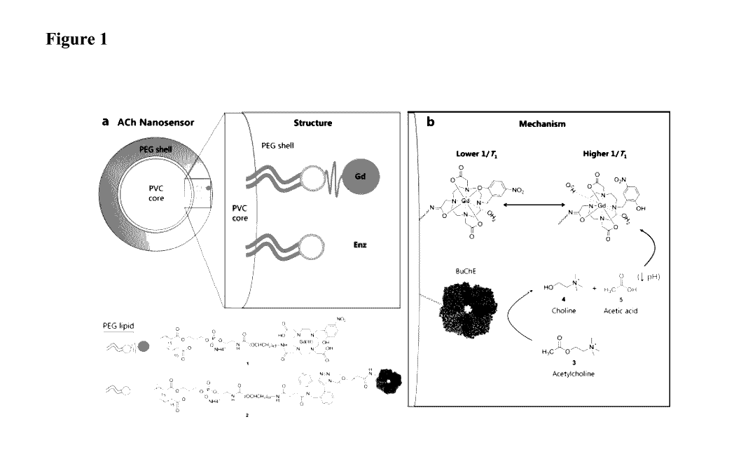

Nanosensors for acetylcholine require a low limit of detection and high sensitivity, due to the low physiological concentration of acetylcholine (nanomolar to micromolar in the extracellular space). To overcome this challenge, in our sensor design, pH-sensitive contrast agents and cholinesterase were immobilized on a platform of nanoparticles that serve as transduction and actuation moieties, respectively. Exemplary embodiments of the invention are illustrated in FIG. 1 and FIG. 19. The mechanism is based on the enzymatic hydrolysis of acetylcholine into choline and acetic acid, and the resulting reduction in local pH alters water coordination of the pH-sensitive contrast agents leading to a decrease in T₁ (increase in r₁). This process is reversible and dependent on the concentration of acetylcholine. The conjugation of enzymes to nanoparticles would increase the proximity between the pH-sensitive contrast agents and the origin of the pH changes created by cholinesterase catalyzed hydrolysis; thus, these nanosensors would detect small local changes in the concentration of acetylcholine.

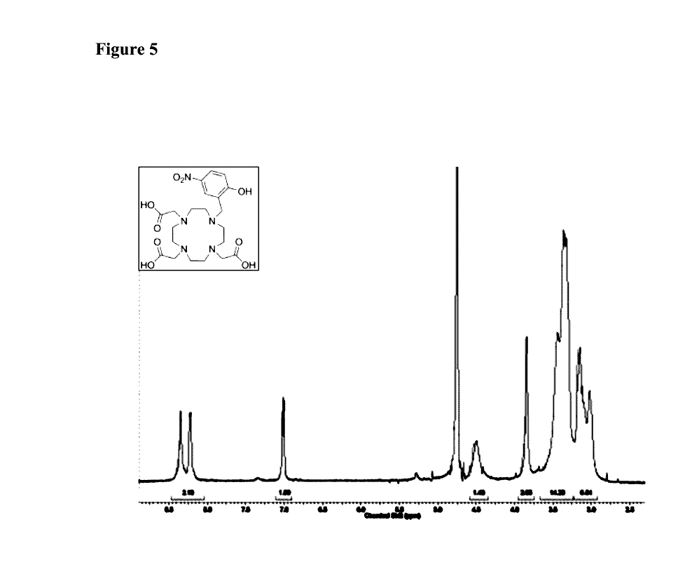

A series of polyvinyl chloride (PVC) or polymer-based nanoparticles were developed to optically sense physiological analytes such as sodium, potassium, lithium, glucose, and acetylcholine. Derivatives of DSPE-PEG lipids were used to form the surface of the nanoparticles with the PVC core (FIG. 1, Part A), where both pH-sensitive contrast agents (Compound 1) and butyrylcholinesterase (BuChE) (Compound 2) were covalently linked to these DSPE-PEG-lipid derivatives. The enzyme was attached to the surface of nanoparticles via copper-free click chemistry in mild conditions to preserve activity of the enzyme. The contrast agent was conjugated to nanoparticle via an amide bond. Among pH-sensitive contrast agents with different mechanisms, in this study, Gd(NP-DO3A) was synthesized and used (FIG. 5), a pH-sensitive analogue of the clinically-used Gd(DOTA) developed by Woods and colleagues. In this structure, paramagnetic Gd(III) was chelated within a twelve-member ring containing a p-nitrophenol. Under basic conditions, only one water molecule is involved in the coordination, while under acidic conditions created by the hydrolysis of acetylcholine, two water molecules will coordinate to Gd, leading to changes in r₁ and T₁ relaxation rate (1/T₁) (FIG. 1, Part B).

To identify the r₁ of ACh-MRNS in PBS, pH=7.4, inductively coupled mass spectrometry (ICP-MS) was used to quantify the amount of Gd bound on the surface of the nanoparticle. With the concentration of Gd(III) and T₁ collected from a 7 T Bruker Biospec MRI scanner for small animals, the corresponding r₁ was obtained. At pH 7.4, the r₁ of the nanosensors was 7.35 mM⁻¹ s⁻¹, which was more than twice the clinically used DOTA-Gd and the free Gd(NP-DO3A) from data obtained in the same scanner (Table 1). Compared to free molecules, the contrast agents on nanoparticles tumble slower, which may account for the improved r₁.

TABLE 1
Relaxivity (r₁) of contrast agents used in this study.

| Contrast agent | r₁ (mM⁻¹ s⁻¹) at pH 7.4 |
|---|---|
| DOTA-Gd | 3.21³⁸ |
| Gd(DO3A-NP) | 3.15 ± 0.47 |
| ACh-MRNS | 7.35 ± 0.73 |

*Errors were calculated from S.D. of T₁ of three independent tests using error propagation.

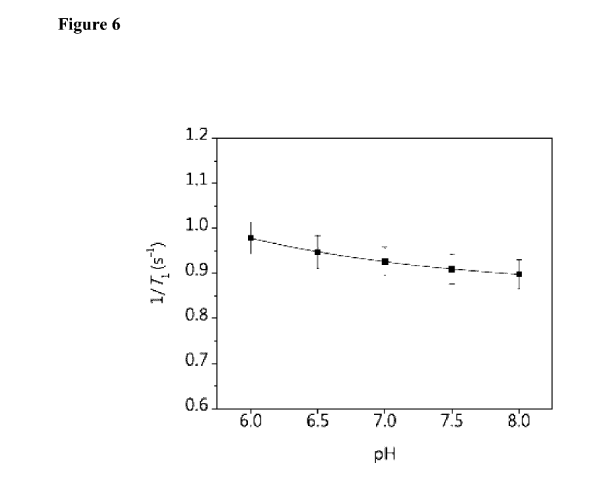

To determine the pH-dependence of the T₁ signal, ACh-MRNS was suspended in PBS, pH 6, 6.5, 7, 7.4 and 8, and scanned using the same MM scanner. The ACh-MRNS demonstrated a moderate sensitivity to pH changes within the physiological range (FIG. 6). When pH increased from 6 to 8, T₁ relaxation rate (1/T₁) dropped by 8%. The extracellular pH in the brain is ˜7.3⁴⁰ and within the pH range 7.2 to 7.8, the 1/T₁ of nanosensors changed by less than 3%. The moderate sensitivity enables ACh-MRNS to respond to local changes in pH, while minimally affected by the global pH fluctuation in the brain.

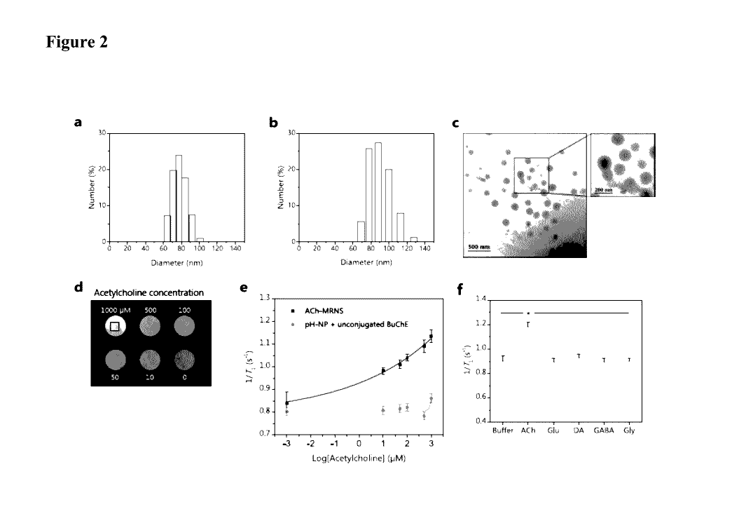

The size and surface charge of fabricated nanoparticles were characterized by dynamic light scattering (DLS) and zeta-potential, respectively. Without conjugation of the enzyme (FIG. 2, Part A), the average hydrodynamic diameter of nanosensors was 77±7.9 nm (mean±standard deviation) and surface charge of −29±3.4 mV. While the conjugated sensors (FIG. 2, Part B) have size of 87±12 nm and surface charge of −41±1.9 mV. Transmission electron microscopy (TEM) in FIG. 2, Part C showed spherical nanosensors with a size of 120±25 nm, which was consistent with results obtained from DLS.

### Example 2: Analytical Calibration and Characterization of the Sensor

The nanosensors were calibrated by suspending an amount of nanoparticles corresponding to 18.9 nmol conjugated Gd(III) in 250 μL solution with concentrations of acetylcholine varying from 0 to 1 mM. A clear gradient in brightness was observed from the T₁-weighted MR image (FIG. 2, Part D): the higher concentrations of acetylcholine led to increasingly brighter images, which represents a higher 1/T₁. A calibration curve of 1/T₁ as a function of concentration of acetylcholine (FIG. 2, Part E), showed an enhancement of 1/T₁ by more than 35%, when the concentration of acetylcholine increased from 0 to 1 mM (blue line). In comparison, the control study using an equivalent amount of pH-sensitive nanoparticle with unconjugated BuChE (pH-NP) led to no significant change in 1/T₁, when the concentration of acetylcholine was increased from 0 to 1 mM (grey). These results indicate that it is necessary to co-immobilize the pH-sensitive contrast agents and enzymes on the nanoparticle in order to achieve sensitivity to micromolar levels of acetylcholine.

The basal level of acetylcholine was anticipated to be within high nanomolar to low micromolar range in the extracellular space in the living brain. Microelectrode studies using either nicotine or KCl stimulation demonstrated to evoke micromolar increases in acetylcholine levels from neurons. Calculation from exponential fitting curve of 1/T₁ (FIG. 2, Part E) in our study displayed that ACh-MRNS can detect difference of ±5 μM when the concentration of acetylcholine is about 11 μM, which suggests the required sensitivity for use in vivo.

Since our sensor is enzyme-based, high specificity was expected against other neurotransmitters (FIG. 2, Part F). To verify the selectivity, the nanoparticles were suspended (corresponding to 18.9 nmol conjugated Gd) in 250 μL solutions of either PBS buffer, 0.5 mM acetylcholine, 5 mM glutamate, 5 mM dopamine, 5 mM GABA or 5 mM glycine. The concentration chosen for the selectivity study was more than 1000 times higher than their physiological level. By measuring 1/T₁, only acetylcholine solution led to a more than 30% increase compared to PBS (P=0.0001, F-value=46.05, df=17; ANOVA with Tukey's post-hoc test), and none of the potential interfering neurotransmitters elicited any significant difference (P>0.5). These results are consistent with the assumption that BuChE selectively hydrolyzes acetylcholine, and supports our nanosensors to respond selectively to acetylcholine.

### Example 3: Acetylcholine Detection in the Rat Brain In Vivo

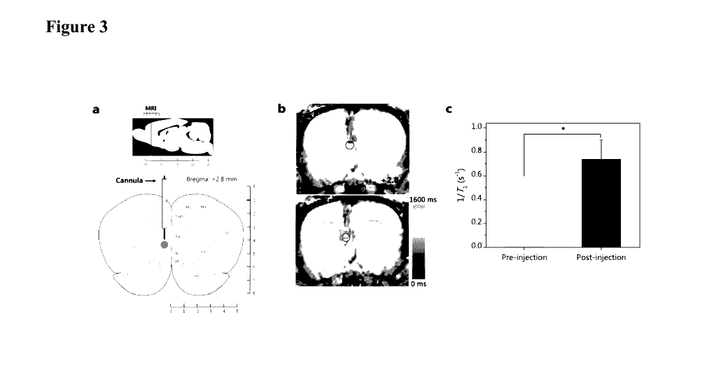

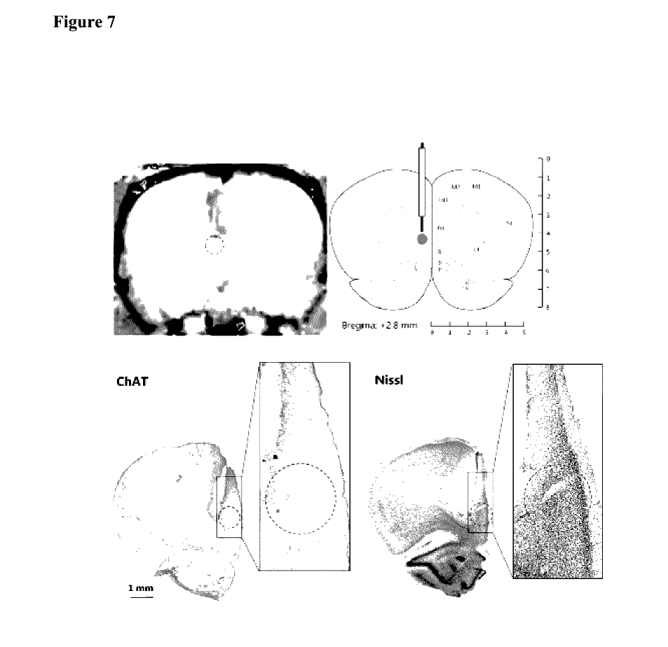

Next, the detection of endogenous acetylcholine release was investigated in the rat medial prefrontal cortex (mPFC). First, to assess the ability of ACh-MRNS to produce reliable contrast in the living brain tissue, 2 μL solution of the nanosensors were injected into the mPFC through implanted cannula of anesthetized rat subjects (FIG. 3, Part A). A strong T₁-weighted contrast was observed at the site of injection (FIG. 3, Part B) that produced an increase of 60% in 1/T₁ (FIG. 3, Part C). This result corresponds to 36±6.2 μM of Gd(III) at injection site, and demonstrates detectable T₁ contrast in the living brain tissue. Histological analysis showed the placement of the cannula in the mPFC (FIG. 7).

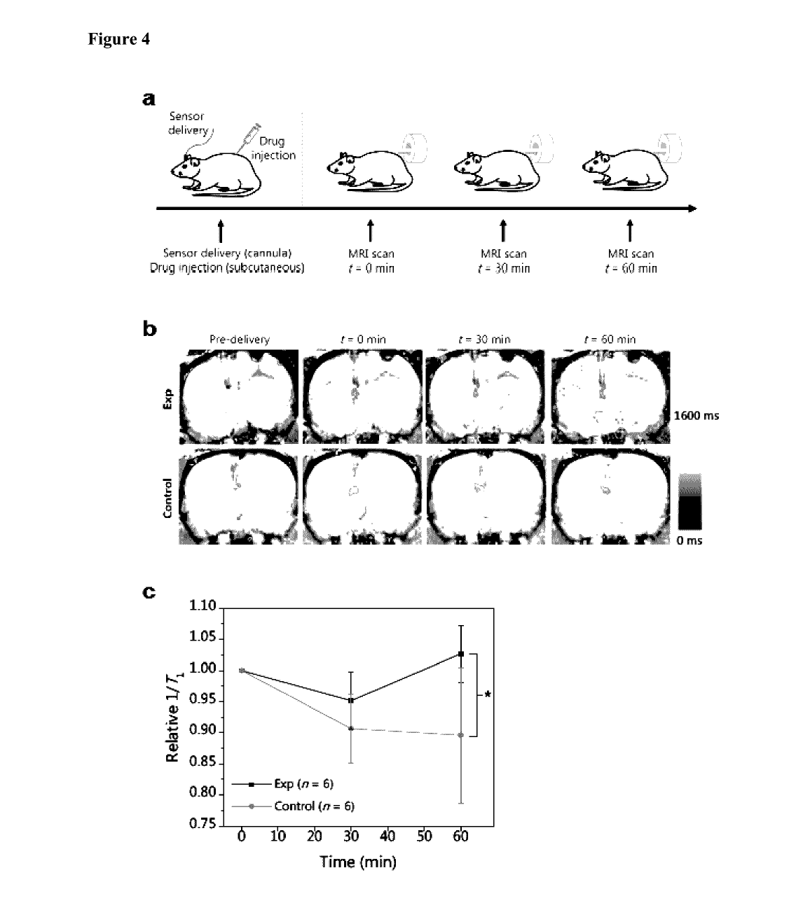

Next, time-course changes in acetylcholine-dependent 1/T₁ were acquired by stimulating the release of acetylcholine in the mPFC using a pharmacological agent, clozapine. Clozapine, an atypical antipsychotic drug, has been shown to induce 2-3 fold increase in acetylcholine concentration from the basal levels, which peaks after 30 minutes, and is sustained for over one hour in the rat mPFC. This experimental paradigm ensures reliable acetylcholine levels can be detected for the duration of the MRI scan procedure. Briefly, the procedure for in vivo imaging included three scans after nanosensor delivery on a 7 T MRI scanner at an interval of every 30 min (MRI scan time points: 0, 30, 60 min post nanosensor injection; see FIG. 4, Part A). Each nanosensor infusion was paired with a subcutaneous injection of clozapine for the experimental group (n=6). For controls (n=6) nanosensors were delivered without clozapine treatment. MRI signal was quantified in regions of interest (ROIs) covering the injection sites. Each ROI volume was defined by a cylinder with a diameter of 1.2 mm and a thickness of 1 mm centered at the injection site, and signal amplitudes were measured and normalized with respect to control ROIs. Examination of the MRI signal time courses in FIG. 4, Parts B-C clearly displayed a difference in 1/T₁ between the duration 30 to 60 min post-injection of nanosensors in the experimental group (blue line) compared to controls (grey line). For both groups, a slight decrease in 1/T₁ was observed at post-injection 30 min period that may be accounted for by nanosensor diffusion. A markedly evident signal change, however, was observed at 60 min with a significant difference of more than 13% 1/T₁ in the experimental group compared to controls (P=0.018, Student's t-test). Other groups have reported that clozapine stimulation has increased acetylcholine concentration by more than 2.5 fold from basal levels (high nanomolar to low micromolar) in the mPFC. Other stimulants, such as KCl, demonstrated an increase of up to 25 suggesting that clozapine-evoked release of acetylcholine, as indicated by a signal enhancement of ˜13% at 60 min in our study, would be detected in the rat mPFC.

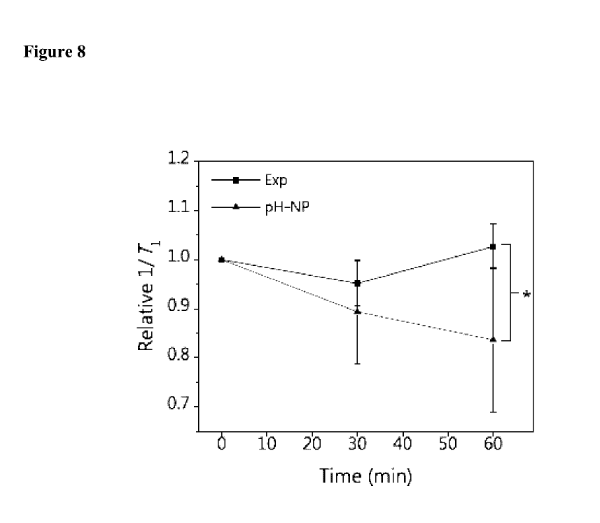

To ascertain whether a variable effect may result from interference with global pH changes in the brain induced by clozapine administration, and not due to local pH changes driven by detection of acetylcholine levels by the nanosensors, identical scanning procedures were performed on new cohorts (n=3). In this group, nanosensors fabricated without enzyme conjugation, i.e., pH-NP (red line, FIG. 8) were delivered as well as clozapine identical to the experimental group above. A similar 1/T₁ was observed after injection of nanosensors (0 min and 30 min post-injection period). However, at subsequent time point at 60 min post-injection, 1/T₁ decreased compared to the experimental group (P=0.005). This result indicates that our nanosensors reliably detect acetylcholine levels as governed by the mechanism of the nanosensor, and that clozapine is not generating a systemic change in pH in the brain.

The present work has shown for the first time the detection of acetylcholine in the living brain tissue using MM. The use of MR-active nanosensors to image acetylcholine is particularly attractive for several reasons. First, the platform of a nanostructure incorporates all sensing components together. The close proximity between cholinesterase and pH-sensitive contrast agent creates a localized effect and facilitates detection of acetylcholine. Second, the use of MRI for detection permits deep tissue imaging in the brain that are not limited to surface measurements, in contrast to optical imaging. Third, the current design offers a platform that can be modified to detect other neurotransmitters and physiological analytes, by substituting the enzymes or functional contrast agents to achieve better specificity and sensitivity. Cannula placement was used for delivery of nanosensors into the mPFC. For ongoing applications in animal studies, disruption of BBB using hyperosmotic shock or ultrasound methods which have been used to deliver small molecules and nanoparticles into the brain, will be explored to improve probe delivery. In addition, the opportunity to investigate other brain regions such as the hippocampus and for the major implications in Alzheimer's disease, schizophrenia, and depression may warrant advances in early disease detection when used with these non-invasive delivery methods. Importantly, higher-resolution imaging paired with faster pulse sequences could be performed to characterize signaling at spatial scales <100 μm, and to improve temporal resolution, respectively. Lastly, enhanced sensitivity for detection of acetylcholine can be achieved by exploring a more efficient enzyme such as acetylcholinesterase. These steps will facilitate the application of MRI and nanosensors for chemical imaging of neurotransmitters fundamental to the understanding of brain function and disease.

In conclusion, a novel neurotransmitter-sensitive MR-active nanosensor was developed for the detection of acetylcholine in the brain. Acetylcholine is a neurotransmitter known to play a prominent role in mammalian social behaviors and neural processes that govern cognition and memory. As such, there is a considerable interest for improved methods for real-time detection of acetylcholine in the brain. The ACh-MRNS reported here provide direct detection of acetylcholine in the living brain tissue for the first time using MM, as characterized and implemented both in vitro and in vivo. Importantly, the modular design of these nanosensors suggests that the current platform can be extended to integrate different components to amplify the sensitivity, active monitoring, and detection of other analytes. These advancements in the design of new MRI contrast agents are hoped to further our understanding of the brain and its disease.

### Materials and Methods for Examples 1-3

2-hydroxyl-5-nitrobenzyl bromide, acetic anhydride, bis-(2-ethylhexyl)sebacate (DOS), butyrylcholinesterase (BuChE), clozapine, dopamine hydrochloride, gadolinium(III) nitrate hexahydrate, γ-aminobutyric acid (GABA), glutamic acid, glycine, methanol, N-(3-dimethylaminopropyl)-N′-ethylcarbodiimide hydrochloride (EDC), N,N′-dimethylaminopyridine (DMAP), N,N′-dimethylformamide (DMF), N-hydroxysuccinimide (NETS), N,N,N′,N′-tetramethyl-O—(N-succinimidyl)uroniumtetrafluoroborate (TSTU), potassium carbonate (K₂CO₃), triethylamine (TEA) and trifluoro acetic acid (TFA) were purchased from Sigma Aldrich (St Louis, Mo., USA). 15-azido-4,7,10,13-tetraoxa pentadecanoic acid was purchased from Alfa Aesar. DO3A tert-butyl ester (t-BOC DO3A) was purchased from Macrocyclics (Plano, Tex., USA). Phosphate Buffered Saline (PBS) (1×, pH 7.4) and sterilized 0.9% saline solution were purchased from Invitrogen (Carlsbad, Calif., USA). Hydrochloric acid (1.0 N) and sodium bicarbonate were purchased from Fisher Scientific (Fair Lawn, N.J., USA). 1,2-distearoyl-sn-glycero-3-phosphoethanolamine-N-[amino(polyethylene glycol)-2000] (ammonium salt) (DSPE-PEG-amine) and 1,2-distearoyl-sn-glycero-3-phosphoethanolamine-N-[dibenzocyclooctyl (polyethylene glycol)-2000] (ammonium salt) (DSPE-PEG-DBCO) were purchased from Avanti Polar Lipids, Inc. (Alabaster, Ala., USA), and methylamine vanadate (Nanovan) was purchased from Nanoprobes (Yaphank, N.Y., USA).

### Synthesis of pH Sensitive Contrast Agents

t-BOC DO3A (200 mg, 0.4 mmol) and 2-hydroxyl-5-nitrolbenzyl bromide (360 mg, 2.0 mmol) were dissolved and stirred in a mixture of 2 mL DCM and 2 mL DMF for 1 h at room temperature. After addition of 600 mg K₂CO₃, the resulting suspension was stirred overnight. Supernatant was then collected after centrifugation, reduced by vacuum, and 3 mL TFA was added dropwise at 0° C., then stirred overnight after it was allowed to warm to room temperature. TFA was removed by vacuum and the residue was purified by flash chromatography to yield product (34% after two steps). H NMR spectrum of product (FIG. 5) was compared to verify success of synthesis.

EDC (7.6 mg, 0.04 mmol) and NHS (4.6 mg, 0.04 mmol) in 200 μL 0.1×PBS (pH 6) was added to pH-sensitive chelates (20 mg, 0.04 mmol) in 3 mL 0.1×PBS (pH 6) in 10 aliquots. The solution was stirred for 30 min and then added to a solution of DSPE-PEG-amine (6 mg, 0.002 mmol) in 3 mL 0.1×PBS (pH 7.4). After the pH was adjusted to 7.4, the reaction mixture was stirred overnight at room temperature. Gd(NO₃)₃.6H₂O (36 mg, 0.08 mmol) in 200 μL DI water was added to the resulting solution in 5 aliquots, and the pH of the reaction mixture was maintained between 4 and 6 during the addition. The resulting solution was stirred overnight at 40° C., then diluted to 24 mL with DI water and stored at 4° C.

Fabrication of pH-Sensitive Nanoparticles

DSPE-PEG-DBCO (0.2 mg, 0.067 μmol) in 20 μL chloroform was dried in a glass vial before addition of 4 mL stock solution of DSPE-PEG attached pH sensitive contrast agent (0.33 μmol, pH 6). The mixture was sonicated at 20% amplitude for 30 s to re-dissolve dried DSPE-PEG-DBCO using a Branson digital sonifier (Danbury, Conn.). Following addition of a mixture of 3 mg PVC and 6 mg DOS in 50 μL THF and 80 μL DCM, the solution was sonicated at 20% amplitude for 3 min. The resulting nanoparticle suspension was filtered using Acrodisc syringe filter with 0.45 μm membrane (Pall Cooperation, Ann Arbor, Mich., USA), washed by DI water (1 mL×5) in 100 kD molecular weight cut off (MWCO) Amicon Ultra centrifugal filters (EMD Millipore, Billerica, Mass., USA), and concentrated to 0.1 mL in DI water by the same filters.

The concentrated suspension was treated with 1.5 μL TEA, vortexed for 3 h, then treated with 1 μL acetic anhydride, and vortexed overnight. The resulting nanoparticle suspension was washed (1 mL×5) in 100 kD MWCO spin-filters as mentioned above and then diluted to 0.5 mL with PBS, pH 8.

### Conjugation of BuChE to Nanoparticles

Solution of 15-azido-4,7,10,13-tetraoxa-pentadecanoic acid (30 nmol in 3 μL DMF), TSTU (1.8 mg, 6.0 μmol) and DMAP (1.4 mg, 11 μmol) were all dissolved in 8 μL DMF. The solution was vortexed for 1 h, and added to solution of BuChE (0.3 nmol in 292 μL PBS, pH 8). The mixture was vortexed for another hour, washed (1 mL×5) and diluted to 0.5 mL with PBS, pH 8 in 100 kD MWCO spin-filters as mentioned above. The modified enzyme solution and 0.5 mL nanoparticle suspension were combined and incubated for 72 h at 4° C. The suspension was concentrated to 30 μL with 100 kD MWCO spin-filters as mentioned above before in vitro calibration.

### Particle Sizing and Zeta-Potential Measurements

The conjugated nanoparticles above were characterized for measurement of particle size and zeta-potential by dynamic light scattering (DLS) using a 90 Plus particle size analyzer (Brookhaven Instruments Corporation).

### Inductively Coupled Plasma Mass Spectrometry (ICP-MS)

To identify the relaxivity (r₁) of nanoparticles, we used a Bruker Aurora M90 inductively coupled plasma-mass spectrometer (Bruker Scientific Instruments, Billerica, Mass., USA) to determine the amount of Gd(III) bound to the surface of nanoparticles. Standard solutions were prepared by dissolving Gd(NO₃)₃ in DI water with different concentrations (0, 0.5, 5, 20, 50 μg/L). The concentrated nanoparticle suspension from previous step was diluted by DI water in ratios of 0.2, 2, 8, and 20 μL/L, and compared with the standard solution to identify the exact amount of bound Gd(III).

### Electron Microscopy

Diluted nanosensors (5 μL) were placed on a 300 mesh carbon film coated copper grid (Electron Microscopy Sciences) for 1 min. The excessive liquid was removed by a piece of filter paper. The remaining sample on carbon film was stained using 5 μL of methylamine vanadate (Nanovan) for 1 min, and then the excessive liquid was blotted by a filter paper. After two rounds of staining, the images were acquired at 120 kV accelerating voltage using FEI Tecnai Cryo-Bio 200 KV FEG TEM. Images were analyzed using ImageJ to measure diameter of nanoparticles.

### In Vitro Calibration

In vitro calibration was performed in a Bruker coil with an inner diameter of 7.5 cm. 2 of a nanosensor suspension was added to 248 μL solution of acetylcholine (0, 50, 100, 250, 500 and 1000 μM) in PBS, pH 7.4 in well plates. The well plate was scanned in a 7 T Bruker Biospec MM scanner for small animals (Bruker Scientific Instrument, Billerica, Mass., USA). A T₁-weighted Rapid Acquisition with Relaxation Enhancement with Variable TR (RARE-VTR) sequence (1 slice; 1.0 mm; TE=12.5 ms, TR=70.000, 291.339, 576.019, 975.509, 1650.942, and 5000.000 ms, FOV=40 mm×40 mm; data matrix 64×64) was used to generate a T₁ map in about 9 minutes. To verify that the pH change is a local effect (FIG. 6), 2 μL pH was suspended sensitive nanoparticles without conjugation of enzyme (pH-NP) and 25 units free BuChE in 248 μL solution of acetylcholine (0, 50, 100, 250, 500 and 1000 μM) in PBS, pH 7.4 in well plates. The same sequence was used for the T₁ map.

### Selectivity Studies

Two μL of concentrated nanosensors were suspended in 248 μL of either glutamate (5 mM), dopamine (5 mM), GABA (5 mM), or glycine (5 mM) in PBS, pH 7.4, and scanned with the same coil and sequence used for in vitro calibration. The resulting 1/T₁ was compared with the 1/T₁ of nanosensors in PBS, pH 7.4 and acetylcholine solution (0.5 mM in PBS, pH 7.4).

### Animal Care and Stereotaxic Surgery

Adult male Sprague-Dawley rats (230-300 g) were obtained from Charles River Laboratories (Wilmington, Mass.). The rats were maintained on a 12:12 h light:dark cycle and allowed access to food and water ad libitum. All procedures were approved by the Northeastern University Institutional Animal Care and Use Committee and were in accordance with the National Institutes of Health guidelines.

Three days prior to MM experiments, unilateral implantation of 26-gauge plastic guide cannula (Plastics One) aimed at the medial prefrontal cortex (mPFC) was performed on animals using a stereotaxic device (Kopf Instruments) under isoflurane anesthesia. A small incision was made to expose the dorsal surface of the skull and wiped clean to reveal the position of lambda and bregma landmarks. A small hole was drilled into the skull at the coordinate position necessary to gain access to the prefrontal cortex (bregma: +2.8 mm anterior, +0.8 mm lateral, +4.0 mm below the surface of the skull). The cannula was placed in the brain and anchored using plastic screws and dental acrylic. The head-wound was then sutured closed and topical antibiotic ointment was applied to the wound area. Buprenorphine (0.5 mg/Kg) was administrated to reduce pain.

### In Vivo Nanosensor Injection and MRI

Animals were first anesthetized with 1-2% isoflurane and placed in a plastic positioning device and a head holder built-in with quadrature transmit/receive volume coil. Infusion of nanosensors into the mPFC was performed by lowering and placing the internal cannula attached to a 10 μL Hamilton syringe via polyethylene tubing filled with nanosensors through the guide cannula, delivering a final volume of 2 μL of nanosensors. The air was first removed prior to nanosensor delivery. The internal-injector cannula protruded 1 mm beyond the guide cannula toward the mPFC.

After delivery, MRI experiments were conducted using a 7 T Bruker Biospec 300 MHz MRI scanner for small animals (same as above for in vitro studies). The design of the positioning device and head holder coil provided complete coverage of the brain from olfactory bulbs to brain stem with excellent B₁ field homogeneity. At the beginning of each imaging session, a high-resolution anatomical data set was collected using the RARE-VTR sequence (25 slices; 1.0 mm; TE=12.5 ms, TR=513.05, 800, 1400, 2200, and 6000 ms, FOV=40 mm×40 mm, data matrix 128×128) to assess time-lapse nanosensor response, followed by acquisition of same sequence at multiple time-points (0, 30, 60 minutes post-nanosensor injection). Each scan took about 20 minutes. For detection of drug-evoked cholinergic transients, subcutaneous injection of clozapine into the back of rat was administered at the time of nanosensor injection, i.e., just prior to T₁ scan at 0 min time point.

### Histological Analysis

To verify cannula placement following MRI contrast agent injection experiments (FIG. 7), animals were anesthetized with carbon-dioxide and transcardially perfused with a solution of PBS (pH 7.4) with 1% sodium nitrite, followed by 4% wt/vol paraformaldehyde in 0.1 M phosphate buffer (pH 7.4). Brains were removed, postfixed for 90 min in perfusion fixative, and cryoprotected in a series of 20% and 30% sucrose in 0.1 M phosphate buffer each overnight at 4° C. Coronal sections of 40 μm thickness across a range extending ˜2 mm anterior and posterior to the cannula insertion site was sectioned on a cryostat (Microm HM 550). Standard protocols were used for choline acetyltransferase (ChAT) immunohistochemical and cresyl violet (Nissl) histological staining. Briefly, for ChAT staining, free-floating sections in well-plates were first incubated in 1% H₂O₂/50% methanol solution for 10 min, followed by serum-blocking buffer for 60 min at room temperature. Sheep polyclonal anti-ChAT antibody (ab18736, Abcam) diluted 1:1,000 in immunobuffer containing 1% normal rabbit serum (Ser. No. 16/120,107, Thermo Fisher) in PBS-0.2% Triton-X100 (PBS-T) was applied and incubated overnight at 4° C. The sections were then incubated with horse-radish peroxidase (HRP)-conjugated rabbit anti-sheep IgG secondary antibody (818620, Thermo Fisher) diluted 1:1,000 in immunobuffer for 2 h at room temperature, and developed in 3,3-diaminobenzidine tetrahydrochloride (DAB) solution (34002, Thermo Fisher). The sections were washed between each step (3×5 min) in PBS-T. The sections were then mounted onto 0.5% gelatin/0.05% chrom alum coated glass slides, allowed to air-dry, and dehydrated through a series of alcohols (75%, 85%, 95%, 100% twice 5 min each), cleared with xylene, and coverslipped with Permount (Fischer Scientific, Pittsburgh, Pa.). The sections were viewed with Olympus BX51 light microscope. Sections processed to determine non-specific staining by following the same procedures, but with omission of the primary antibody, showed no immunohistochemical labeling.

### Image and Statistical Analysis

All data analysis and image processing was performed with Bruker Paravision 5.1 software (Billerica, Mass., USA), Matlab (Mathworks, Natick, Mass.), and itk-SNAP. Images were reconstructed and analyzed using custom routines running in Matlab. Relaxivities were calculated from T₁ obtained from itk-SNAP and concentration of Gd(III). Graphs and illustrations were compiled using Origin (OriginLab, Northampton, Mass., USA) and Illustrator (Adobe, San Jose, Calif., USA), respectively.

Errors were propagated from standard deviation (S.D.) of T₁ (σT₁) or normalized T₁ (σ(normalized T₁) using formula derived from previous reports³.

The equation used for Table 1 is defined as:

$$Variance = \frac{σ {{T}_{1} ( average of {T}_{1} )}^{- 2}}{[ Gd ]}$$

where, [Gd]: concentration of Gadolinium in mM.

The equation used for FIGS. 2, 3, and 6 is defined as:

Variance=σT₁(average of T₁)⁻²

The equation used for FIGS. 4 and 8 is defined as:

Variance=σ(normalized T₁)(average of normalized T₁)⁻²

where, normalized T₁: T₁ of each animal normalized to 0 min post nanosensor injection.

P-values to examine selectivity were calculated via one-way analysis of variance (ANOVA) with Tukey's HSD post-hoc test for multiple comparisons. P-values for in vivo data were calculated using Student's t-test. When P<0.05, the difference was considered as significant.

### Example 4: Properties of the pH-Sensitive Contrast Agent

Fluorescent probes were designed that are capable of visualizing acetylcholine release and clearance. There are advantages to this nanosensor construct embodiment. First, sensor components are modular, and new components can change the sensor's target molecule without significantly modifying the remaining components. Second, the system is highly tunable because substituting the types, concentrations, or ratios of individual components changes the sensor's response. Finally, sensor components are protected from fouling by the biocompatible coating that imparts lifetimes from days to several weeks, which compares very favorably to other implantable sensor technology (FIG. 11).

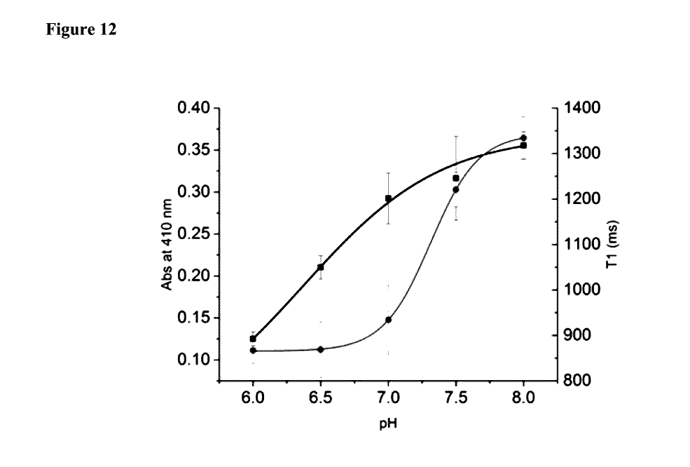

To fabricate the MM sensor for acetylcholine, gadolinium (Gd)-based contrast agent was used to sense pH changes generated by the hydrolysis of acetylcholine. Since the absorption of the contrast agent at 410 nm decreases in acidic conditions, this parameter can be used to identify pKa of the contrast agent during development (FIG. 12). This calibration was used to estimate that the pKa of the contrast agent is around 7, which is close to physiological pH (7.4), and this was a predictive tool for the MR signal. The MR response in acidic conditions gave rise to a higher r1 and a lower T1, and at high pH, r1 was low and T1 was high. The T1 map also indicated a pKa between 7 and 7.5. Note that T1 increased by 45% when the pH changed from 6 to 8 (FIG. 12).

### Example 5: MRI Nanoparticle Fabrication

Polymer-based nanoparticles can sense important physiological analytes such as sodium, potassium, lithium and glucose. Polyvinyl chloride (PVC) was used. As with our previous sensors, DSPE-PEG derivatives formed the surface of the PVC core, and pH sensitive contrast agents were covalently linked to the DSPE-PEG derivatives. The hydrophilic contrast agent oriented towards the water phase, the size of the nanoparticle was around 150 nm, and the surface charge was −27 mV.

### Example 6: Relaxivity of the Nanosensors

To identify the exact relaxivity of contrast agent, ICP-MS was used to measure the amount of Gd located on the surface of the nanosensor. By comparing the abundance of Gd on nanosensor with work curve, the real concentration of Gd and the corresponding relaxivity was calcualted. When actylcholine concentration increased from 0 to 1 mM, the relaxivity was enhanced from 8 to 16.7 mM-1s-1. The relaxivity of the nanosensors is higher compared to clinically used DOTA and the free pH sensitive contrast agent in FIG. 10 (Table 2). The slower tumbling rate may account for this increase. This higher relaxivity guarantees that an efficient contrast can be generated by our nanosensor.

### Example 7: Enzyme Conjugation and In Vitro Calibration of Nanosensors

Nanosensors for acetylcholine require a low limit of detection and high sensitivity, due to the low physiological concentration of acetylcholine (micromolar to 1 millimolar). Conjugating the enzyme to nanoparticles should enhance the proximity between the pH-sensitive contrast agent and the origin of the pH changes. Thus, these nanosensors should be able to detect small changes in the concentration of acetylcholine. To conjugate the enzyme to nanoparticles, copper-free click chemistry was applied. Azide-modified enzyme reacted to DSPE-PEG-dibenzocyclooctyne (DSPE-PEG-DBCO) by incubating under 4° C. for 3 days. Because only mild conditions are required, this reaction did not harm the enzyme's activity. DSPE-PEG-DBCO and contrast agents were both linked to the surface of nanosensors (FIG. 11).

TABLE 2
Relaxivity of DOTA, free pH sensitive contrast
agent and acetylcholine nanosensor

| | Acetylcholine Concentration | Relaxivity |
|---|---|---|
| Contrast agent | (μM) | (mM⁻¹ s⁻¹) |
| DOTA | 0 | 3.5 |
| Free pH sensitive Contrast | 0 | 5.5 |
| agent | | |
| Nanosensor | 0 | 8.0 |
| Nanosensor | 50 | 9.4 |
| Nanosesnor | 100 | 10.7 |
| Nanosensor | 500 | 13.5 |
| Nanosensor | 1000 | 16.7 |

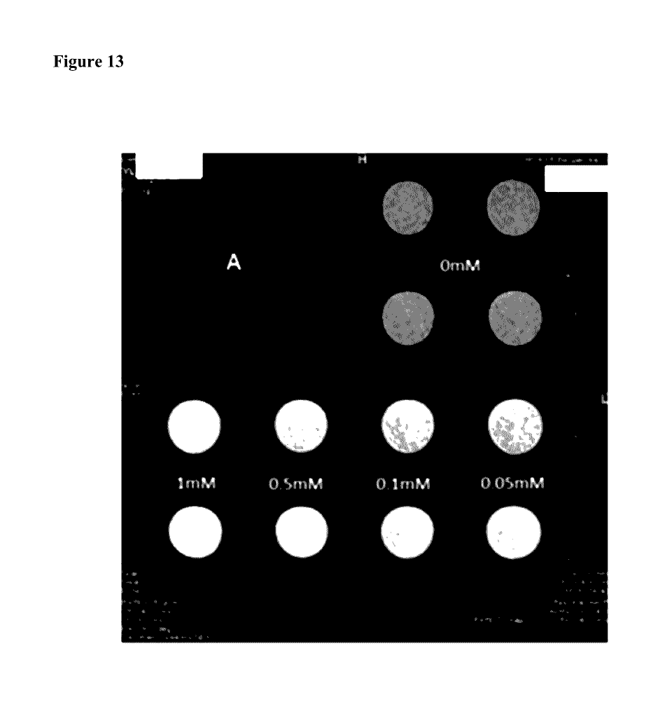

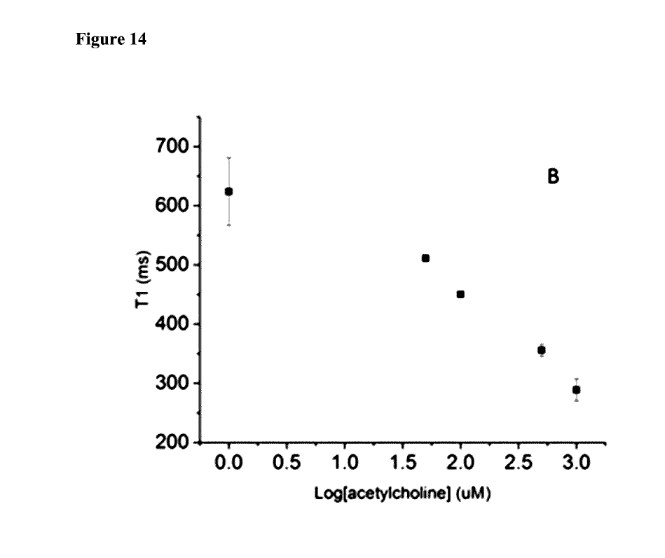

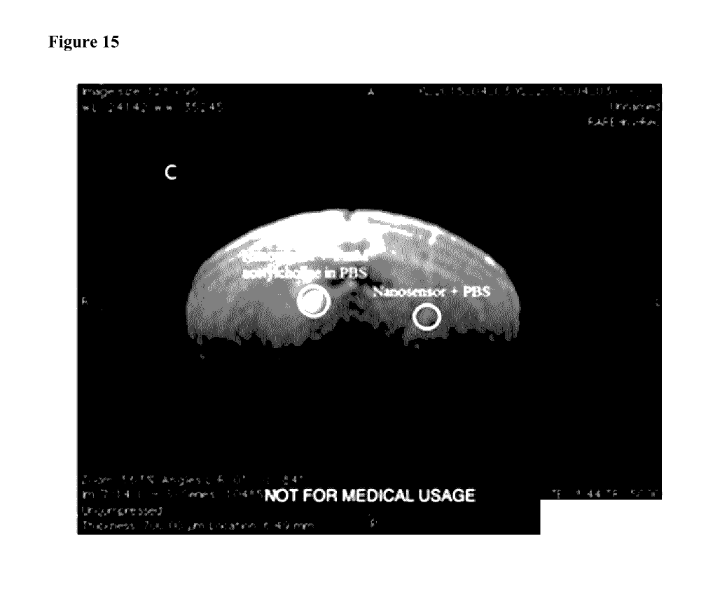

After conjugation, the Kd was measured at 100 μM in the MR scanner, resulting in a clear change in brightness: the higher concentration of acetylcholine led to a brighter image than lower concentration (FIG. 13 and FIG. 14). The success of this in vitro study suggests that our nanosensor will be able to capture a subtle change in physiological range of acetylcholine. An increase in acetylcholine concentration from 0 to 100 μM led to a greater than 30% decrease in T1. This sharp drop should enable application of this technique in vivo.

To verify nanosensors can enhance contrast and respond to acetylcholine in tissue, a mixture of acetylcholine (1 mM) and nanosensors was injected into left hemisphere of a mouse brain. Nanosensors were injected in buffer alone into right hemisphere as a control group. The experiment group generated a brighter image at injection site than the control group, since the relaxivity of the contrast agent increases when acetylcholine is present (FIG. 15). In addition, a fluorescent dye (Rhodamine 18) was incorporated into the nanosensor to confirm its distribution using fluorescence microscope after the MRI procedure was completed.

### Example 8: Injection and Distribution of Nanosensor In Vivo

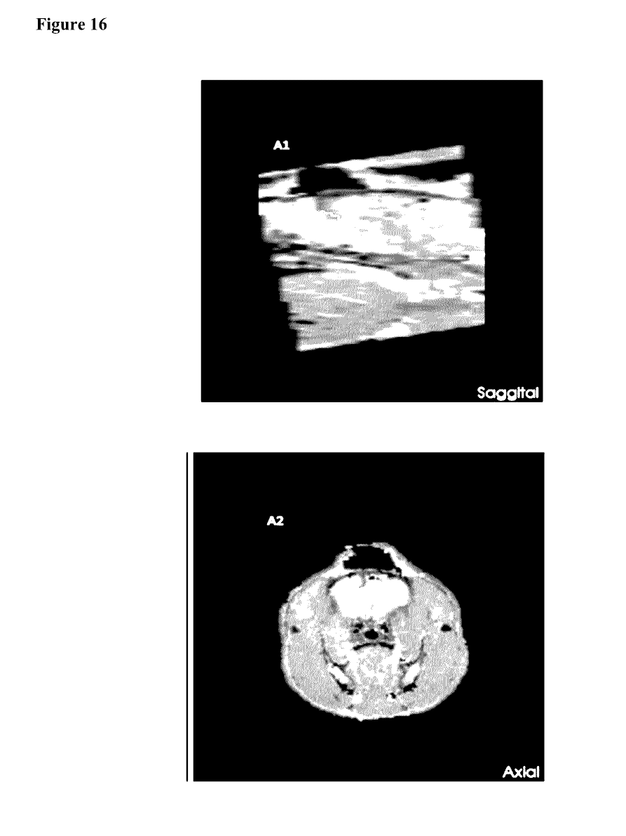

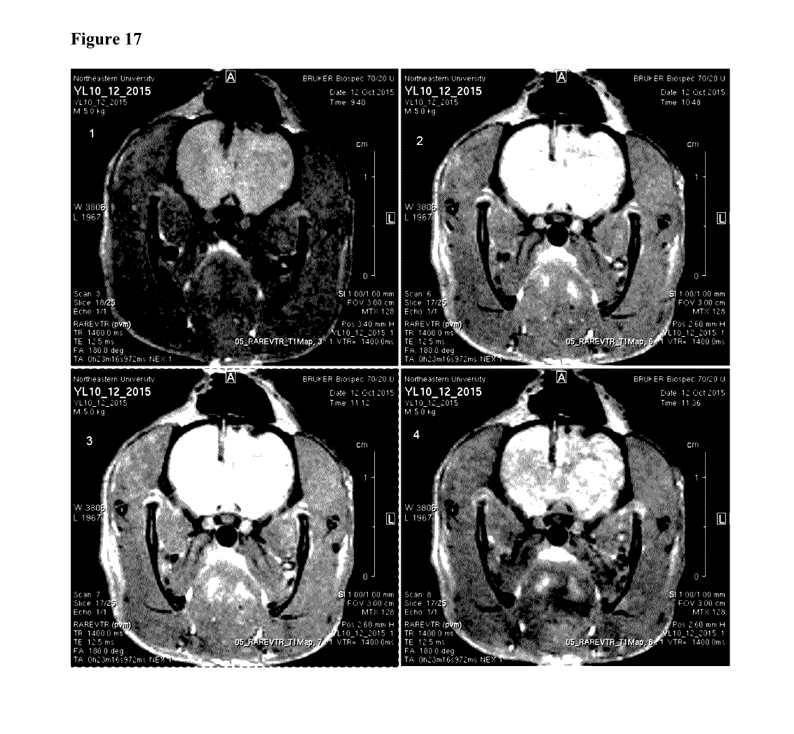

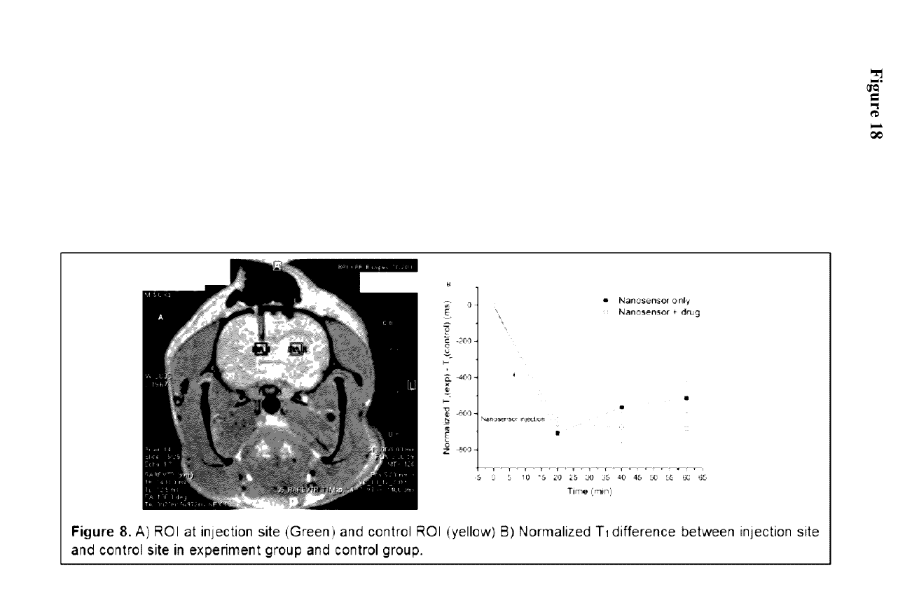

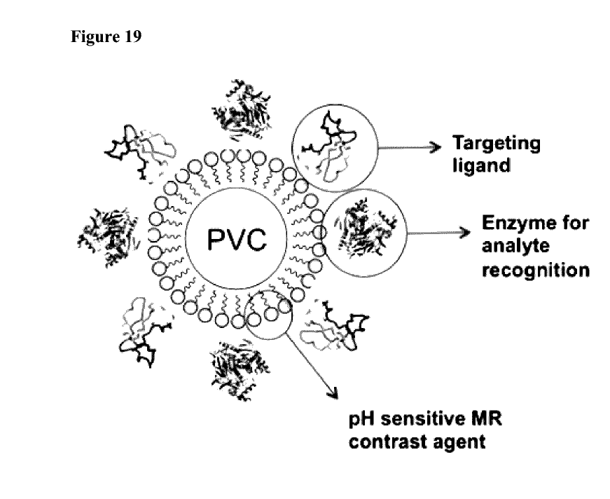

The distribution of nanosensor can be affected by its diffusion. To test the distribution of nanosensor in vivo, a nanosensor suspension was injected to the medial prefrontal cortex of Sprague-Dawley rats via an implanted plastic cannula using stereotaxic surgery (FIG. 16). The cannula pointed to the prelimbic cortex, which is a sub-region of medial prefrontal cortex associated with working memory, time interval discrimination, novelty detection and other behaviors. Image obtained from a 7 T small animal MR scanner (Bruker) indicated that a significant amount of nanosensor remained at the site of injection with minimal diffusion during the first hour of nanosensor injection (FIG. 17 and FIG. 18). This provides sufficient amount of T1 signal to be detected and imaged at the specific site of nanosensor injection.

### Example 9: Nanosensor Response Against Drug Induced Acetylcholine Release

Acetylcholine level can be elevated 2 to 3 fold from the baseline in 30 minutes and maintained for more than one hour without the inhibition of acetylcholinesterase. To test the response of the nanosensors to homogenously released acetylcholine, clozapine was injected subcutaneously to induce aceytlcholine release in the mPFC. The MM scanning procedure included a pre-scan, followed by nanosensor injection into the brain, then drug administration, and finally measurements at three time points. Each scan took 20 minutes (FIG. 17). An ROI was taken right below injection site for T1 calculation and used ROI of same size on the other hemisphere of brain as control (FIG. 18). The plotted curve indicates a sizeable drop in the T1 signal at the nanosensor injection site (more than 30%) while the T1 at the control ROI remain stable during the whole scan. The diffusion of nanosensors may have resulted in a slight increase of T1 in the control group 40 minutes after injection. In the experimental group, this increase due to diffusion was offset by actual sensor response to acetylcholine release. Thus, compared to the T1 of control group, the T1 of experiment group slightly decreased after injection. Another strategy is to perform a nanosensor-only scan before the injection of clozapine to generate a basal level contrast. The nanosensor's response to the drug induced acetylcholine would further decrease the basal level T1. Analysis of signal using voxel based methods will also help to limit error bars and provide accurate comparison between T1 changes in the experiment and control group. Preliminary results indicate that our proposed nanosensors can be imaged in vitro. Our sensors can capture subtle changes in acetylcholine levels in the micromolar range. The nanosensors generated a relaxivity higher than clinically used DOTA. These preliminary data point to the feasibility of using our proposed sensors to detect acetylcholine in vivo. These outstanding in vitro results promise a great potential for future in vivo work. The preliminary in vivo study showed that most of the sensor remained at the injection site for at least 60 minutes after injection. The nanosensor has been successfully injected to the mPFC and a significant reduction in T1 signal has been observed at the injection site. More experiments and improved strategy will be implemented for detection of homogeneously released acetylcholine.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A composition, comprising:
(a) a plastic polymer core;
(b) a shell surrounding the plastic polymer core, wherein said shell comprises a plurality of amphiphilic lipids, each of which comprises a hydrophobic end and a hydrophilic end; and the hydrophobic ends of the amphiphilic lipids are linked to the plastic polymer core;
(c) a ligand comprising a binding moiety, wherein the binding moiety is capable of binding to a binding site present on a transmembrane receptor;
(d) an enzyme capable of catalyzing the hydrolysis of a target compound; and
(e) a contrast agent;
wherein the ligand, the enzyme, and the contrast agent are conjugated to the hydrophilic ends of the amphiphilic lipids.

2. The composition of claim 1, wherein the plastic polymer core consists of polyvinyl chloride (PVC).

3. The composition of claim 1, wherein the plurality of amphiphilic lipids comprises 1,2-distearoyl-sn-glycero-3-phosphoethanolamine-N-(polyethyleneglycol)(DSPE-PEG).

4. The composition of claim 1, wherein the transmembrane receptor is located on a synaptic membrane.

5. The composition of claim 4, wherein the transmembrane receptor is an acetylcholine receptor.

6. The composition of claim 1, wherein the ligand is a ligand for an acetylcholine receptor.

7. The composition of claim 1, wherein the ligand is a protein.

8. The composition of claim 7, wherein the protein is a bungarotoxin.

9. The composition of claim 1, wherein the target compound is acetylcholine.

10. The composition of claim 1, wherein the enzyme is a cholinesterase.

11. The composition of claim 10, wherein the cholinesterase is butyrylcholinesterase.

12. The composition of claim 10, wherein the cholinesterase is acetylcholinesterase.

13. The composition of claim 1, wherein the contrast agent is pH sensitive.

14. The composition of claim 1, wherein the contrast agent comprises gadolinium (Gd).

15. The composition of claim 1, wherein the contrast agent comprises 1,4,7,10-tetraazacyclododecane-1,4,7,10-tetraacetic acid (DOTA).

16. The composition of claim 1, wherein the contrast agent is DOTA-Gd.

17. The composition of claim 1, wherein the contrast agent is GdNP-DO3A (gadolinium 1-methlyene-(p-NitroPhenol)-1,4,7,10-tetraazacycloDOdecane-4,7,10-triAcetate).

18. The composition of claim 1, wherein the composition is substantially spherical.

19. The composition of claim 18, wherein the diameter of the composition is about 50 nm or less.

20. The composition of claim 1, wherein the plurality of amphiphilic lipids comprises DSPE-PEG, the ligand is a ligand for an acetylcholine receptor, and the enzyme is a cholinesterase.

21. The composition of claim 1, wherein the plurality of amphiphilic lipids comprises DSPE-PEG, the ligand is bungarotoxin, and the enzyme is a cholinesterase.

22. A method of imaging a target compound, comprising
(1) administering to a subject an effective amount of a composition of any one of claims 1 to 21; and
(2) obtaining a functional magnetic resonance image (fMRI) of the subject, thereby imaging the target compound.

23. The method of claim 22, wherein the target compound is acetylcholine.

24. The method of claim 22, wherein the plurality of amphiphilic lipids comprises DSPE-PEG, the ligand is a ligand for an acetylcholine receptor and the enzyme is a cholinesterase.

25. The method of claim 22, wherein the plurality of amphiphilic lipids comprises DSPE-PEG, the ligand is bungarotoxin, and the enzyme is a cholinesterase.
